(19) United States
(12) Patent Application Publication
(10) Pub. No.: US 2026/0045337 A1
(43) Pub. Date: Feb. 12, 2026

(54) SYSTEMS AND METHODS FOR GENERATING HEALTH CARE REFERRALS BASED ON EPISODES OF CARE TRACKED USING PROPRIETARY MARKERS

(71) Applicants: Michael Joseph Tambe, Holliston, MA (US); Raymond Basri, Middletown, NY (US)

(72) Inventors: Michael Joseph Tambe, Holliston, MA (US); Raymond Basri, Middletown, NY (US)

(21) Appl. No.: 18/799,813

(22) Filed: Aug. 9, 2024

Related U.S. Application Data
(63) Continuation of application No. 15/145,427, filed on May 3, 2016, now abandoned.
(60) Provisional application No. 62/156,291, filed on May 3, 2015.

(51) Int. Cl. G16H 15/00; G06Q 40/08; G16H 10/60

(52) U.S. Cl. CPC G16H 15/00; G06Q 40/08; G16H 10/60

(57) ABSTRACT

A method of using data markers in a health insurance entity claims database to assist in tracking episodes of care (EOCs) to assist in generating referrals of medical specialists to a referring user, comprises providing an interface enabling a referring user to request a medical procedure for a patient and for making a referral of a medical specialist to assist with the medical procedure; identifying medical specialists; accessing a health insurance entity claims database storing historical claims associated with the same medical procedure; evaluating the historical claims to retrospectively estimate historical EOCs; using a respective data marker in the health insurance entity claims database to uniquely mark each retrospectively estimated historical EOC; using predictive analytics to generate an expected EOC for each medical specialist; generating an expected cost and a list of candidate medical specialists therefrom; receiving a selection; generating and using a particular data marker to mark an actual claim for the particular medical procedure and actual claims for the actual subsequent medical events to track the actual EOC in the health insurance entity claims database.

### TECHNICAL FIELD

The present invention relates generally to a patient care provider referral system. Specifically, the present invention provides healthcare providers, including primary care physicians, specialists, nurse practitioners, physician assistants, chiropractors, and administrators with a mobile application that creates, manages, and monetizes the referral. Particularly, the system and method of the present invention uses predictive analytics to enable insurance companies, physicians, and their patients to understand the expected patient-specific cost of a specialty referral based on policy restrictions, fee schedules, participating network providers, and related data. The system and method then recommend a specialist provider based on the patient specific cost, overall quality, and patient specific travel distance of the specialty provider.

### DESCRIPTION OF THE RELATED ART

There exist in the related art a number of methods and systems which facilitate the ordering, tracking, and management of the specialty consultations and diagnostic testing referrals made by a referring physician.

For example, there exist in the art, checklists that direct a patient to a particular provider or for a particular type of treatment based on rules and evidence algorithms. In such a system, physicians are directed by a checklist of steps, based on patient symptoms, and the checklist directs the primary care physician as to whether or not to make a referral or prescribe a certain treatment.

In another internet-based system, the checklist determines whether or not the referral should be made based on the data entered by the primary care physician.

The prior art also contains systems that simply manage referrals. For instance, one system is a network of interconnected computers submitting a referral request by a first provider, accepting the request by a second provider, obtaining insurance approval and obtaining and updating the medical record of the patient with that information.

US Patent Application Number 20130325509 discloses a method/software whereby the referring physician may access a database and input search criteria to obtain a list of providers and, then, refer the patient to one or more of these specialists. US Application '509 also discloses an electronic health record (EHR) tracker configured to track updates to an EHR of the patient.

US Application 20140304003 discloses a referral analytics methodology that can yield a list of physicians or specialists within a set geographic area who are properly specialized in, and/or have a history of managing, the condition, activity, procedure, or diagnosis. The selected physicians may be filtered by patient preferences and default preferences.

U.S. Pat. No. 8,571,889 discloses a mechanism for accounting for attribution and referrals. The total cost of each episode of care is determined as a result of the choices made by the provider and, in particular, a specific physician or person who has control over the costs associated with that episode of care. The difference between the total costs for the specific episode of care, compared to the predetermined baseline value for that episode of care, equals the resulting savings that have been realized. The resulting savings is then split between a provider, the payer, and the incentive administrator.

None of the prior art methods and systems are dynamic and robust in their ability to update their databases in real-time or be responsive to patient or payer constraints regarding the costs of the services.

Accordingly, there exists a need for a system and method of ordering, tracking and managing referrals which is able to update its database in real-time. Further, there exists a need for a method and a system to manage the referral as a commodity to effectively incentives the referring physician.

Moreover, there exists a need for a method and system for referrals which provides cost transparency at the post of care, not simply by quality of care and geographical distance. Furthermore, there exists a need for a method and system of referrals enabling the authorization criteria to be customized by each payer and can also be adjusted to accommodate the needs of the entity authorizing the referral, whether it is the payer or network of providers.

There further exists a need for a method and system which is capable of being linked and/or integrated with telemedicine modules so that a patient can complete part or all of specialty referral remotely.

There also exists a need for a method and system that keeps referrals within a network of providers.

There further exists a need for a method and system that links to an appointment scheduling module of the specialist to allow for instantaneous scheduling and confirmation of the procedure desired between the referring physician and the specialist physician.

There moreover exists a need for a method and a system which enables a referring physician or specialist to concurrently prescribe and/or order medications, durable medical equipment, home care and nursing services for the patient.

### SUMMARY

An advantage of the present invention is that it provides a system for creating a patient care referral, which includes a database of records for a plurality of users, a database of current procedural terminology codes, a database of a patient's records, a database of a payer's records, a processor, a memory operably coupled to the processor and storing software executable by the processor, the software including a referral creation means configured to retrieve a current procedural terminology code for a procedure from the current procedural terminology code database, a search means configured to enable a referring user to select a patient from the patient's records database, a payer payment means to enable the referring user to determine the costs to the patient for the referral, an authorization means to enable the referring user to determine approval by the payer for the referral, a confirmation means to enable the referring user to confirm the referral with the payer, a messaging means to enable the referring user to communicate with the selected patient care provider or the patient or the payer; and a payer records database updating means enabling the payer record database to be updated to reflect the referral.

An additional advantage of the system of the present invention is that the authorization means allows the insurance provider to authorize the referral in real time.

Another advantage of the system of the present inventions that the confirmation means confirms the referral in real time.

A further advantage of the system of the present inventions that the messaging means enables the patient care provider to communicate with the user or the patient or the payer.

Another advantage of the system of the present invention is that the messaging means enables the payer to communicate with the user or patient or patient care provider.

An additional advantage of the system of the present invention is that the messaging means communicates with a selected party in real time.

A further advantage of the system of the present invention is that the payer records database updating means updates the payer records database in real time.

Another advantage of the system of the present invention is that the updating means updates the patient's health insurance costs information.

Another advantage of the system of the present invention is that it provides for a dynamic patient care provider database, which includes a database including records for a plurality of patient care providers, an access means enabling a user to access the patient care provider database, a cost variance means enabling the user to enter a high value and a low value for its service into the patient care provider database, an expected cost for the providers services based on the patient's medical condition, negotiated rates with different insurance providers, and patients medical history, a plurality of metrics to indicate the quality of care the patient care providers provides.

An additional advantage of the system of the present invention is that for each specialty of patient care the system can calculate the cost variance within the market and, for each patient care provider, the variance of their services.

A further advantage of the system of the present invention is that a shared saving payment to either the referring physician using the app or the patient receiving care can be calculated based on the relative cost of the provider selected versus market rates.

A further advantage of the system of the present invention is that it can display information that the specialist provider authors to the referring physician to aid his referral decision.

An additional advantage of the system of the present invention is that the dynamic database includes a query means enabling the user to query the patient care provider database for costs of service and a query response means capable of responding to a query for costs of service on behalf of the user;

An additional advantage of the system of the present invention is that when the dynamic patient care provider database is queried, the systems can weight cost, quality, and travel distance for patient to recommend a specific provider the patient should utilize

An additional advantage of the system of the present invention is that the recommendation algorithm can be configured to prefer patient care providers within a health insurance providers network, thereby reducing cost of care.

A further advantage of the system of the present invention is that the dynamic database also includes an analysis means enabling the user to analyze a query response of another patient care provider.

Another advantage of the system of the present invention is that it enables a user to analyze a selection process for a patient care provider, in that it provides a database including queries for records for a plurality of patient care providers, a tracking means enabling a patient care provider to track the number of times it has been queried for costs for a procedure selected for a procedure, and an analysis means enabling the patient care provider to analyze the database using predetermined criteria.

A further advantage of the system of the present invention is that it provides a user with the ability to tracking an episode of care by creating a patient care referral and assigning a numeric marker to the patient care referral, entering the numeric marker for all subsequent patient procedures incidental to the referral; and a tracking means enabling a user to track the patient procedures from referral to completion.

### BRIEF DESCRIPTION OF THE DRAWINGS

These and other features and advantages of the present invention will become more readily appreciated as it is better understood by reference to the following detailed description when considered in connection with the accompanying drawings, wherein:

FIG. 1 is a schematic representing an overview of the method and system of the present invention;

FIG. 2A is a schematic depicting the method and system for creating a referral of the present invention;

FIG. 2B is a schematic depicting the method and system for creating a referral of the present invention;

FIG. 3 is a schematic representing the method and system for monetizing the referral of the present invention;

FIG. 3A is a screen shot depicting monetization of the referral of the present method and system;

FIG. 4 is a schematic representing the method and system of tracking an episode of care of the present invention;

FIG. 5 is a schematic representing the method and system of the present invention for creating a dynamic patient care provider database of the present invention;

FIG. 6 is a screenshot of the application of the method and system of the present invention depicting the Account Management dashboard available to an Authorized User;

FIG. 7 is a screenshot of the application of the method and system of the present invention depicting the manner in which an Authorized User may update a referral object with claims data;

FIG. 8. is a screenshot of the application of the method and system of the present invention depicting the manner in which an Authorized User may also view all referral objects and see the current status of all referrals made by an individual Physician using filters by procedure family and date;

FIG. 8A is a screenshot of the application of the method and system of the present invention depicting the Procedure Families available to the Authorized User for the query;

FIG. 8B is a screenshot of the application of the method and system of the present invention depicting a query for all dates and all Procedure Families;

FIGS. 9A, 9B, 9C, 9D, 9E and 9F are screenshots of the application of the method and system of the present invention depicting use of the Messenger function;

FIG. 10 is a screenshot of the application of the method and system of the present invention depicting Procedure Families;

FIG. 10A is a screenshot of the application of the method and system of the present invention depicting the manner in which an Authorized User may view or modify Procedure Families;

FIG. 11 is a screenshot of the application of the method and system of the present invention depicting Participating Physicians;

FIG. 11A is a screenshot of the application of the method and system of the present invention depicting the manner in which an Authorized User may upload Participating Physician data;

FIG. 12 is a screenshot of the application of the method and system of the present invention depicting Rated Specialists;

FIG. 12A is a screenshot of the application of the method and system of the present invention depicting the manner in which an Authorized User may add an individual Rated Specialist to the database;

FIG. 12B is a screenshot of the application of the method and system of the present invention depicting the status of the bulk upload of the Rated Specialists an Authorized User may add Rated Specialists in bulk to the database;

FIG. 12C is a screenshot of the application of the method and system of the present invention depicting the manner in which an Authorized User may add an individual Rated Specialist to the database;

FIG. 13 is a screenshot of the application of the method and system of the present invention depicting the manner in which an Authorized User may view or modify Physician Groups; and

FIG. 13A is a screenshot of the application of the method and system of the present invention depicting the status of the upload of the Physician Group an Authorized User may add to the Physician Group database.

### Definitions

The following are Terms and Definitions utilized in the present application and the example provided herein:
(a) Authorized user: A user who is registered on the website and successfully authenticated in the system. This user has privileges to see “Account Management” dashboard. Authorized users are representatives of Payers.
(b) Claim: An HCFA 1500 standardized form for the billing of medical services by a Provider. This is the basic transaction of health services used within the application. Claims are unique to each user of the easyrefmd.org application and claims data (.csv format) is used for uploading Claims into easyrefmd.com site.
(c) Current Procedural Terminology Database: A database of all Current Procedural Terminology (CPT) codes from the American Medical Association. All active procedural codes are enabled in this database
(d) easyrefmd.com: The webpage for the tablet application for the method and system of the present invention.
(e) easyrefmd.org: The home webpage for the method and system of the present invention.
(f) Global Physician Database: A database of Providers from a trusted third party service such as https://ppiregistry.cms.hhs.gov/.
(g) Groups: Both Participating physicians and Rated specialists may organize themselves into groups in their contractual relationships with the Easyrefmd.com application user (an insurance provider).
(h) Guest user: A user who opened EasyrefMD.com site and is not authenticated in the system. This user has no privileges to see “Account Management” dashboard.
(i) Insurance provider (Payer): A representative of Insurance Company (an Insurance Provider, or payer), Easyrefmd.com end user.
(j) Local Databases: These include the following databases: CPT, Participating Physicians, Payer, Patient, Groups, Procedure Family, Claims, Referrals.
(k) National Provider Identifier (NPI): A unique 10-digit identifier to allow Patient Care Providers to identify themselves in a standard way throughout their industry. Individuals or organizations apply for NPIs through the CMS National Plan and Provider Enumeration System (NPPES). After receiving an NPI, NPPES publishes the parts of the NPI record that have public relevance, including the Provider's name, specialty (taxonomy) and practice address.
(l) Participating/Referring Physicians: A unique practicing physician in the United States. These physicians will be end users of the easyrefmd.org tablet web application.
(m) Patient: An individual seeking medical care. Each Patient has a unique ID.
(n) Patient Care Provider (PCP): A unique practicing physician in the United States. Each PCP has a unique NPI. These physicians will be end users of the easyrefmd.org tablet web application.
(o) Patient Care Provider Database: A database containing the names of Patient Care Providers and their specialty of practice, location of office, office hours, types of insurance accepted, and costs for procedures
(p) Patient Database: A database containing the Patient's identifying information, insurance coverage and medical records, among other things.
(q) Patient Payer Database: A database which contains information regarding the Patient's insurance plan(s). This database includes information such as plan name, plan number, insurer identification number, patient information, deductible limits, co-pay requirements, and secondary insurance providers. It further includes the Patient Care Provider Database.
(r) Patient Payer Record: A record of Claims covered and/or paid by the Payer on behalf of the Patient, including remaining deductible, co-payments applicable, etc.
(s) Payer: An insurance company or other entity responsible for paying the Claim.
(t) Payer Database: A database of Payers.
(u) Primary Care Physician: The Patient Care Provider originating the Referral.
(v) Procedure Family: A synthesized description of a specialty medical procedure such as “Colonoscopy”, or “Nuclear Stress Test”. It is a specialty procedure at the level an average doctor or patient would understand. The Procedure Family is unique to each user of the easyrefmd.com website application.
(w) Rated Specialists: A list of recommended Patient Care Providers for the Referral. These physicians are specialized and do not need to be registered users of easyrefmd.org web application themselves.
(x) Referral: A recommendation made by a Patient Care Provider for a Patient to be evaluated and treated by a Rated Specialist. A Referral may be categorized as follows:
   Pending: A submitted Referral that has not yet been fully completed. Once a tablet app user submits a Referral, it is considered pending.
   Completed: A Referral that has been successfully followed through by both Patient and Patient Care Provider and is ready for patent by the Payer.
   Paid: A Referral that has been paid for by the Payer.
   Expired: A Referral that is not followed through by the Patient within 90 days of the Referral Date.
(y) Referral Date: The date and time stamp indicating when a referral was created.
(z) Referral Value: The payable value of the Referral.
(aa) Shared Savings Payments: The scheme of monetizing the referral as disclosed in the present method and system.
(bb) Specialist Provider/Physician: A table of pointers to Patient Care Providers in the Patient Care Provider Database physicians who will be displayed as recommended providers by the application.
(cc) Specialist Provider Ratings: A table evaluating the Claims data to generate a rating for each Patient Care Provider. This will show cost, quality, and address data for each Specialist.
(dd) Specialist Ratings: For the algorithm to display recommendations, it is necessary to have cost, quality, and address data for each specialist. It should be noted that most of this data will be derived from and/or validated against claims data.

### DETAILED DESCRIPTION OF THE PREFERRED EMBODIMENTS

The subject invention comprises a method and system that provides Referring Physicians with a mobile application that provides analytics, benchmark fees and utilization rates for physicians, specialists and testing facilities.

The method and system of the present invention may also be used to provide services via telemedicine. For example, Primary care and specialist referral services may provide a consultation with a patient using live interactive video or store and forward transmission of diagnostic images, vital signs and/or video clips along with patient data for later review. There may also be remote patient monitoring, including home telehealth, uses devices to remotely collect and send data to a home health agency or a remote diagnostic testing facility (RDTF) for interpretation. Patients will also have available to them specialized health information and online discussion groups to provide peer-to-peer support via the Internet and wireless devices.

### The System for Creating a Referral

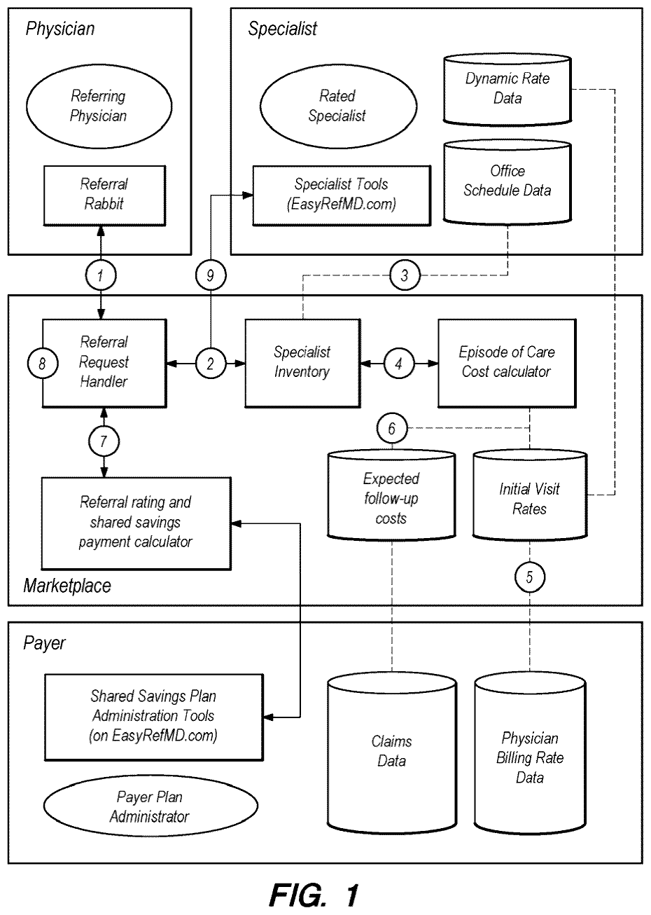

As shown in the overview in FIG. 1, A Referral request is submitted on the Referral Rabbit tablet application by a Referring Physician and is received by the EasyRefMD Marketplace Request Handler. The Marketplace Request Handler queries the specialist inventory to find all available specialists for the requested specialty service within patient's network, geographic distance, and schedule availability. The inventory is refreshed by receiving available times from specialists who have integrated their appointment booking system with the system.

For all available specialists in the inventory, the expected cost for an Episode of Care is retrieved for each specialist from the Episode of Care cost calculator. The Episode of Care cost calculator retrieves initial visit data from both the Payer's negotiated rate card and any updated rates the specialist wishes to offer. The Episode of Care cost calculator also estimates follow-up costs (e.g., second visit, further prescriptions) from the Payers historical claims data.

Once the Referral Request Handler receives available specialists and cost, it then analyses the referral rating and shared savings payment calculator to rate the available specialists and determine the shared savings payments. The Referral rating and shared savings payment calculator queries payer data from shared savings program information and physician quality to obtain this data.

The Referral request handler compiles a list of available specialists and delivers the results on the Referral Rabbit app where they are displayed for the Referring Physician for his selection. Once a referral is completed the event is logged and notification is given to the specialists.

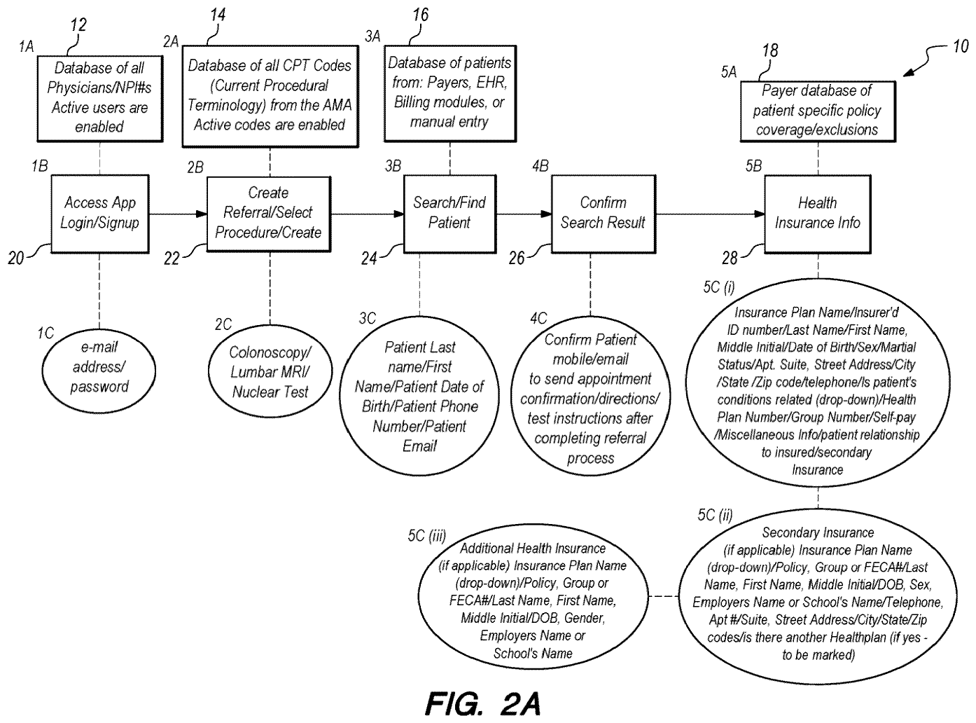

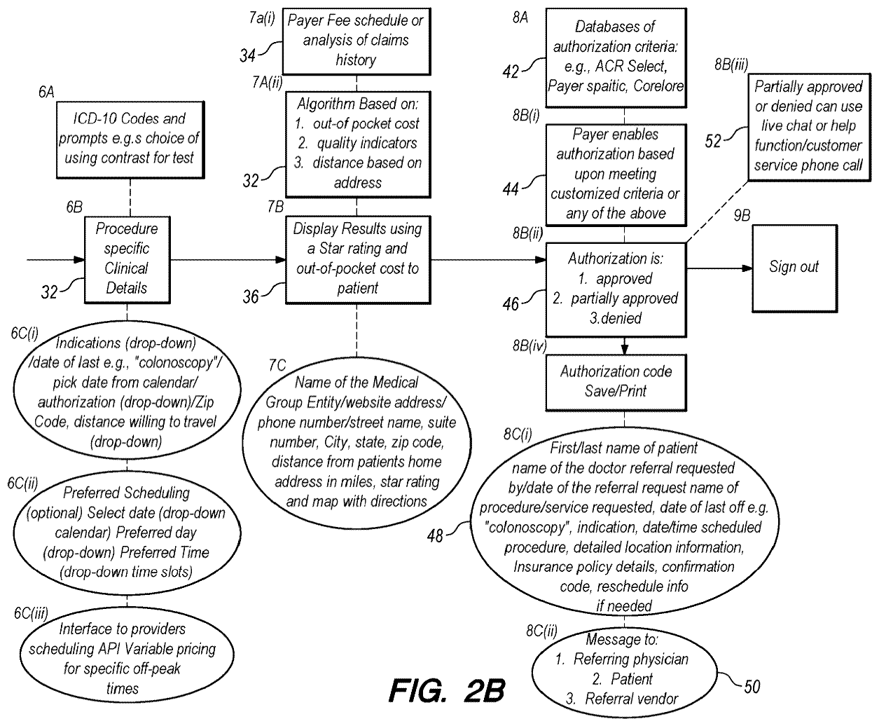

With reference to detailed FIGS. 2A and 2B, in a preferred embodiment, the system of the present invention includes the following components: a User Database 12, a Current Procedural Terminology Database (CPT) 14, a Patient Database 16, a Payer Database 18. The databases may be stored in one location or a plurality of locations, and it is understood that both the local and non-local databases are able to be communicated with by conventionally known means and are stored on a computing device which has a processor and a memory operably coupled thereto and capable of executing the software instructions to be executed by the processor. The databases are able to be accessed by the user, generally a Primary Care Physician (PCP), a payer, a Patient Care Provider (Provider) or other authorized individual or entity using computing devices such as PC's, tablets, smartphones or any other compatible device. These devices may be used to access databases that are local or through a server using the internet. In addition, the method and system of the present invention can be used within an Electronic Health Record which imports and exports data to populate the app or as a standalone web application

The CPT Database 14 is a database of all CPT codes from the American Medical Association (AMA) and all active codes are enabled in this database.

The Patient Database 16 includes information about the patient's identifying information insurance coverage and the patient's medical records, among other things.

The Patient Payer Database is a database which contains information regarding the patient's insurance plan(s) and includes information such as plan name, plan number, insurer identification number, patient information, deductible limits, co-pay requirements, and secondary insurance providers. It further includes a Patient Care Provider Database, which is a database containing the names of Patient Care Providers and their specialty of practice, location of office, office hours, types of insurance accepted, and costs for procedures. Each patient has a unique Patient Payer Record, and all Patient Payer Records are located in the Patient Payer Database. This record is created by a PCP when a new patient is accepted by the PCP and is generated in conjunction with the Patient Payer.

As depicted in FIG. 2A, a Login function 20 allows an active user 12, to access the system of the present invention. A Create Referral function 22 allows the user to access the CPT Database and choose the appropriate code for the procedure desired. A Search function 24 allows a user to access the Patient Database to locate the Patient who is the subject of the referral. A Confirm function 26 allows a user to confirm that the patient whose record is being displayed is in fact the correct patient for whom a referral is being sought. Once it is apparent that the correct patient's record is able to be accessed, the patient's Payer database 18 will be accessed and the patient's health insurance information 28 will be available for view, if desired. The health insurance information includes the plan name, plan number, the insured's identifying details, such as first name, last name, date of birth and gender. It further includes the patient's marital status, address, phone number, self-pay details, patient's relationship to the insured, and information regarding secondary insurance. The health insurance information most importantly includes the payer's patient specific policy coverages and exclusions.

Subsequently, the user is prompted to enter any details specific to the procedure that is being prescribed 30. Following the full entry of information regarding the procedure, the algorithm 32 of the present invention provides results 36 based on the payer fee schedule or analysis of the claims history for the patient 34 and the out of pocket costs to the patient, the quality indicators for a Patient Care Provider and Geographical distance of Patient Care Provider to Patient. However, it is to be understood that any of these metrics may be altered based upon preference. The results display will also indicate the amount of monies the Payer will pay for the procedure and informs the patient of the out-of-pocket costs. A specific recommendation of a provider fulfilling the criteria input by the referring physician will be generated by the method and system of the present invention. The results generated are able to include additional information authored by the specialty provider, e.g., promotional material from the specialist, links to the provider's website, etc. Further, the specialist may offer variable pricing based on availability, time of day, or modification of a network fee schedule, perhaps by an adjustable coefficient to a fee schedule, enabling a specialists to fill all of their available time slots.

A Selection Means 40 allows the user to select a Provider. Once the Provide has been selected, the Authorization Means 42 allows the user to begin the process of obtaining authorization from the Patient Payer for the referral. The authorization is based on an analysis of the Payer's database of authorization criteria 42 and allows the Payer to enable or disallow authorization based upon the analysis of its criteria 44. The results of this analysis of the authorization criteria are displayed to the user. 46. If the procedure is authorized, the Confirmation Means 48 allows the user to confirm the procedure with the Provider and schedule an appointment for the Patient, thereby creating the referral.

The Messaging Means 50 allows the user to message the Provider, Patient and Payer regarding the confirmation, scheduling, results and any other items related to the Patient and procedure. A Patient may also consult with the specialist or referring physician via telemedicine.

If the procedure is not authorized or only partially authorized, the user may communicate with the Payer via live chat or telephone regarding the denial 52. The Payer may provide the Patient of Referring Physician with web-based links to request additional data or input to make the authorization via chat, email, text, or phone.

Following the completion of the creation of the referral, the patient payer records are updated in real time to reflect the scheduling of the procedure and the changes to deductibles.

Confirmation that the procedure was completed occurs when the specialty provider submits a claim. The claims data is input into the system and verified. Upon verification, the referral will be marked as completed and a shared savings payment paid to the referring physician. The results of the procedure are emailed to all parties upon request for claims data, allowing the user and patient to view and discuss the results of the procedure as quickly as possible and from remote locations, if necessary, thereby obviating the need for the patient to schedule a follow-up appointment in person with the referring user. With regard to the payments to the Referring Physician and Provider, the payment for the procedure is sent directly to them by the payer. The payment to the Referring Physician is considered to be the monetization of the referral, and is detailed herein below. In the event that a patient needs or requests a procedure that is not specifically covered by their policy, the Payer may be able to offer the Referral to the Patient with a variable cost to the Patient depending on the degree of authorization or policy specific coverage available. For example, a Patient wants genetic testing for cancer but has no benefits within their policy. The Payer may make a partial payment if the Patient is willing to pay the remainder.

### Method of Monetization of the Referral

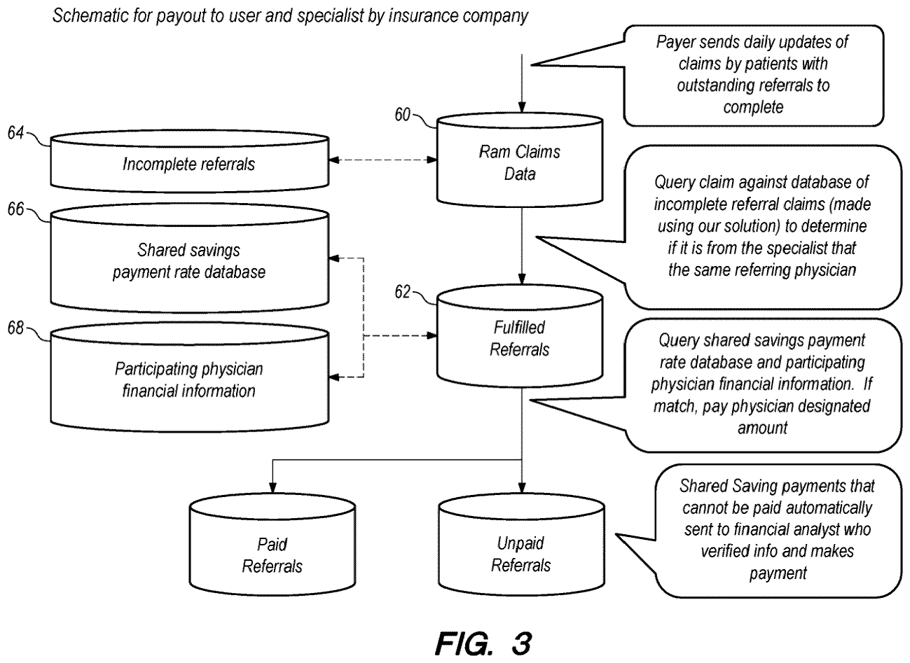

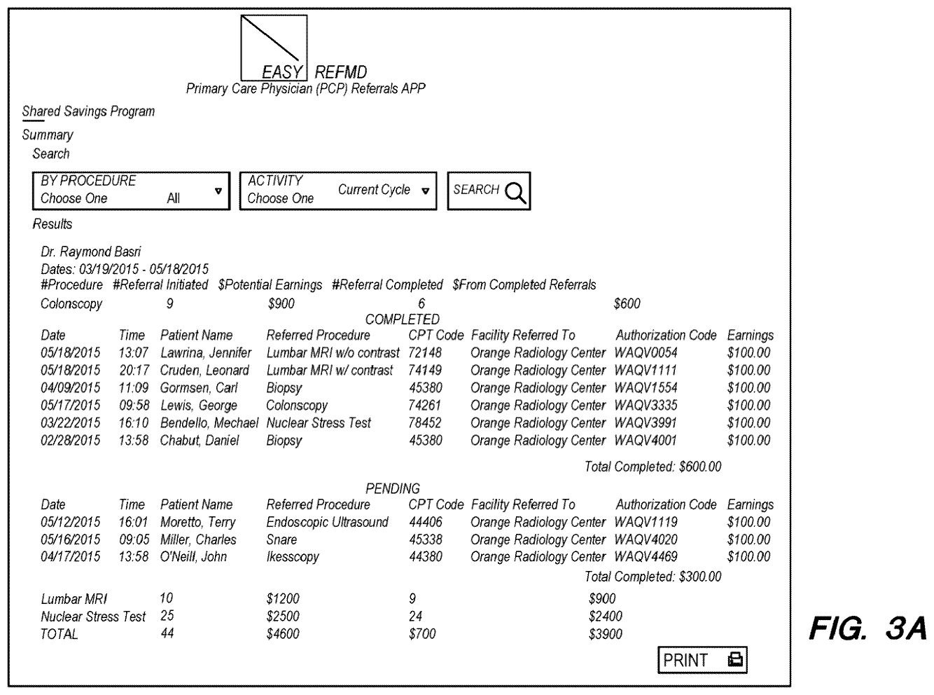

With reference to FIG. 3, the referral made by the Referring Physician is monetized to further incentivize the Referring Physician to use the method and system for creating a referral of the present invention. This monetizing is also referred to as the Shared Savings Payment. Referrals created utilizing the method of the present invention populate the database of Raw Claims Data 60. Referrals may be categorized as fulfilled 62 or incomplete 64. The fulfilled referral database 62 includes information from the payment rate database 66 and the participating physician information database 68. By utilizing the method and system for creating a referral of the present invention, the rate of fulfilled referrals is dramatically increased due to ease of creating the referral, reduction of costs to patient and timely payment to all involved. As depicted in FIG. 3A, fulfilled referrals are monetized for the Referring Physicians, thereby motivating the Referring Physician to create referrals utilizing the method and system of the present invention. Specifically, Shared Savings Payments may be calculated based on the cost of the selected providers versus a market rate. For example, if there are three doctors in a 20 mile radius capable of performing the specialty service, then the method and system of the present invention will evaluate the cost and patient volume for all three, with the insurance provider and calculate market rate as a weighted average. If Dr A charges $2000 and sees 20% of the Payer's patient, Dr B charges $800 and sees 40% of the Payer's patients and Dr C charges $1200 and sees 40% of the Payer's patients, then the market rate=40%*$800+40% $1200+20%*$2000=$1200. Hence choosing Dr A provides a $400 savings, a percentage of which will be paid to the Referring Physician for the Referral.

### Tracking an Episode of Care

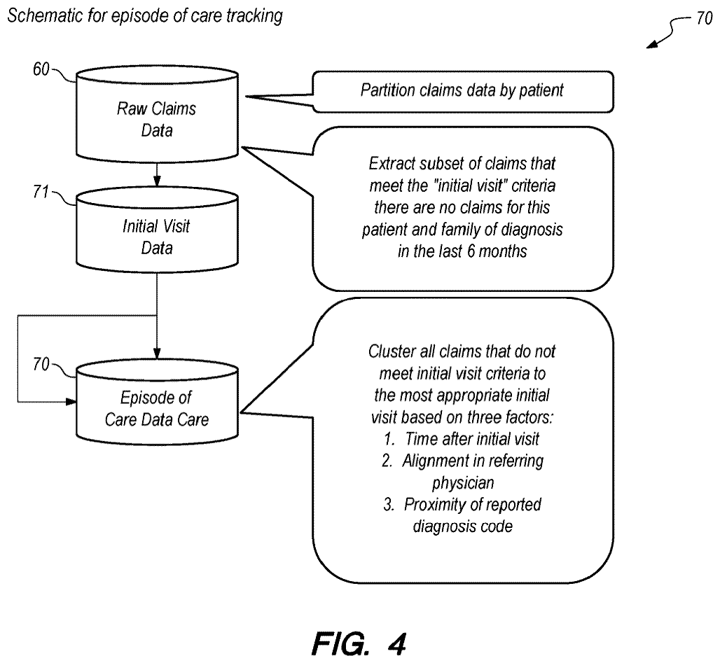

As depicted in FIG. 4, the system of the present invention has also created a proprietary marker to track an Episode of Care (EOC) 70 beginning with the symptom or finding, including routine health maintenance, triggering the first PCP referral. In order to mark and track an EOC 70, it is necessary to review the patient's health care data 60 and extract all data that meets the criteria of initial visit 74. The initial visit is defined as “no claims for the patient and the diagnosis for the patient within the past 6 months”. Once the initial visit has been determined, it is marked using a numerical code. Subsequently, all referrals and care based on the initial visit are also marked with the numerical code signed to the initial visit and tracked with the marker. This addresses a key shortcoming in the methodology payers currently use to calculate an episode of care (EOC). Previously an EOC was measured from the last significant procedure working backward to identify a cluster of claims for services. Therefore, an EOC would lack accuracy for both the symptom at first presentation and concurrent conditions also requiring referrals. The system of the present invention enables early opportunity to influence the PCP as the first referral is made thereby bending the cost curve for the EOC dramatically.

The method and system of the present invention also improve the Payer's Medical Loss Ratio (MLR) so that they are able to more accurately assess their expected payout, and, thereby, will more accurately predict their reserve requirements.

### Creating a Dynamic Patient Care Provider Database

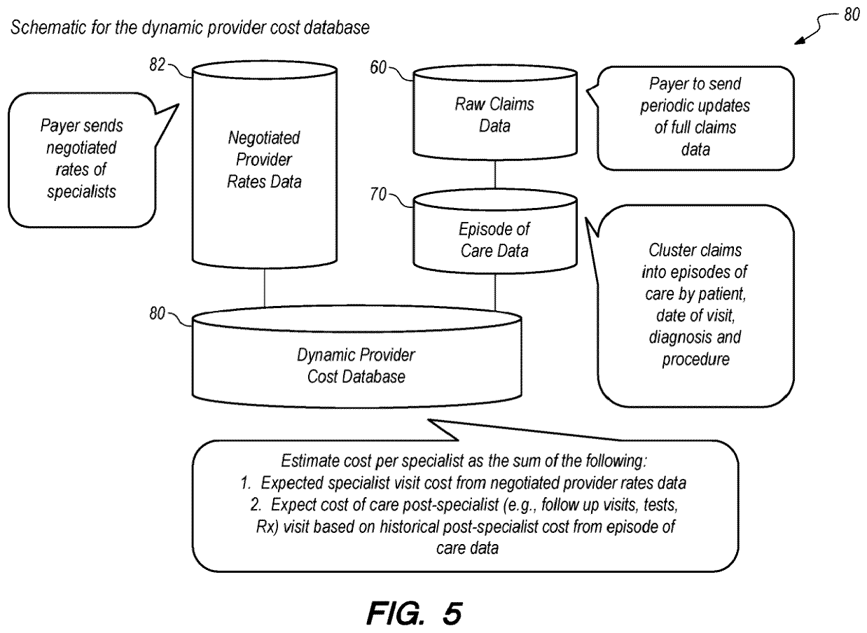

As depicted in FIG. 5, the present invention also provides a system and method to create a dynamic Patient Care Provider Database 80. This dynamic Database 80 is populated using raw claims data 60, Episode of Care (EOC) data 70, and provider rates data 82. The dynamic Database 80 calculates and estimates the cost per specialist as the sum of the expected specialist visit cost as indicated from the provider rates data 82 plus estimated care of cost from the post specialist provider based on the EOC data 70. The provider rates data 82 may be provided by the payer based on previously known or negotiated rates between the payer and provider. The claims data 60 may, also, be provided by a payer. The EOC data 70 is a proprietary marker and is generated as detailed above In use, an authorized individual from the Patient Care Provider will have access to the Patent Care Provider Database which is maintained by the Payer. This user will be enabled to enter a floor price and floor savings for each procedure or test or item offered by their organization. The system and method of the present invention will, at the point of referral, share this data with other Providers and disclose each aspect and breakdown of the cost.

By providing this level of transparency and clarity, the system will allow Providers to modify their cost proposals for procedures based on other participants'economics. A user may also query the Patient Care Database for the costs of particular procedures or tests or items offered by other users in order to modify its own floor price offering to the Payer. Furthermore, the system and method of the present invention will allow the user to query the Patient Care Provider Database to obtain an analysis of the number of times and reasons it has not been chosen to be the provider for a particular referral.

The method and system of the present invention will also perform a retrospective claims analysis to set a baseline for cost and utilization. This baseline cost will be calculated as follows:
Expected cost=Negotiated rate of specialty visit
   +Probability a follow up visit required×negotiated rate of specialty visit
   +(Probability of follow up test or prescription
   ×cost of said follow-up test or prescription)
   +all likely follow-up tests and prescriptions

### Example: Monetizing the Referral/Shared Savings Payments

The Shared Savings Payment is the mechanism by which the Payer monetizes the Referral, and, thus, incentives Physicians to utilize the method and system of the present invention. Accordingly, the Payer must be able to ascertain whether a given Referral is valid and has been completed. Specifically, every time the Authorized User uploads a claim, that claim is compared to the set of all pending and expired claims to see if there is a match. This is accomplished via the Account Management dashboard.

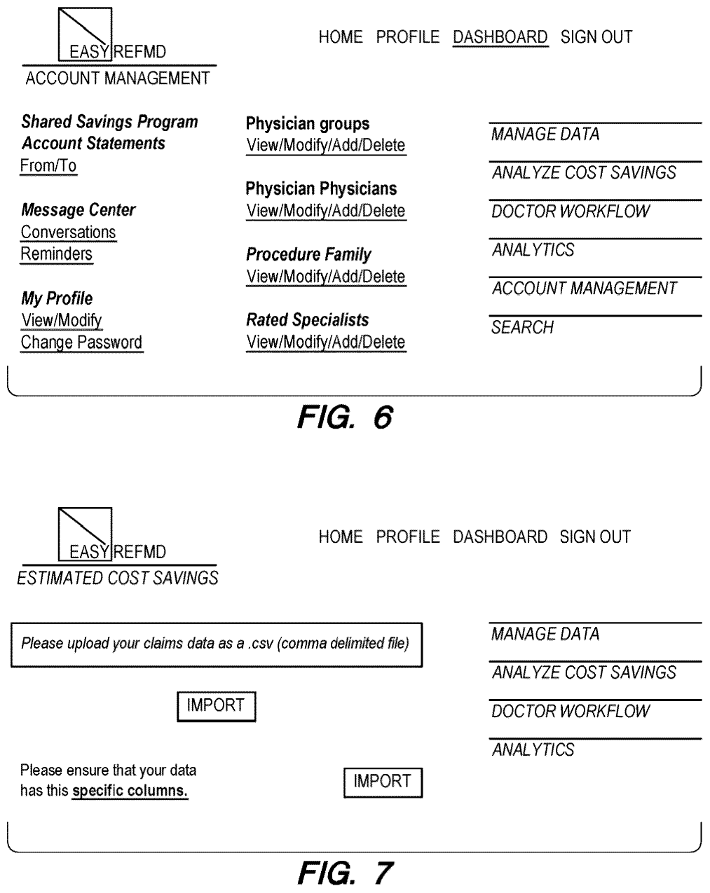

With reference to FIG. 6, there is depicted the Account Management dashboard. The Account Management dashboard shows all possible options for users who successfully gain authorization into the system. For the Shared Savings Payments, a Payer is the only Authorized User, although there are 2 types of users for the EasyrefMD.com website: Guest user and Authorized user. An Authorized User gains access to this screen by clicking the Account Management icon and entering their user login. Consequently, an Authorized User is redirected to the “Account Management Dashboard” page, FIG. 6. The Account Management Dashboard includes the following Active links and Working processes:
Shared Savings Payments/Account Statements:
Message Center
My Profile
Physician Groups
Participating Physicians
Procedure Family
Rated Specialists

The Account Management dashboard also allows and Authorize User to Manage Data, Analyze Cost Savings, review Doctor Workflow, run Analytics and Search the various databases.

When a registered Physician completes a referral on the Referral Rabbit Tablet Application, that Physician creates a referral object. A referral object contains the following pieces of data:
1. Referring Physician NPI
2. Patient Member ID
3. The procedure family referred
4. Referral Date—date and time stamp when referral was created in the tablet app.
5. Referral Value—a dollar amount: this value must be populated based on data in the application. Specifically it is a referral value=(efficiency—1)*the market rate*payment percentage (this should be a global variable that can be set on the backend (no need to frontend at this point, set it as 5%). If this number is negative, the value is set to zero.
6. Referred Specialist—the NPI of the physician or the Group ID of the physician group whom the doctor is referring (i.e., which option the tablet app user selected when seeing a range of options on the last screen).
7. Referral Status—This field can have three values, however, when a referral is created, its default value is Pending.
8. Completed Claim—this starts as null, but will eventually contain a pointer to a record of the claims database.
9. Completed Date—default=null. Will eventually contain the date from the claim to which completed claim points.
10. Paid Date—Default=Null

Once the referral has been made, an Authorized User may update a referral object with claims data, FIG. 7. As can be seen in the Fig., claims data to be uploaded is a .csv file. The .csv file should have the same fields which are listed in the “specific columns” section at the easyrefmd.com website, specifically, the following:
1. Member ID—The unique identifier of the patient who is being referred
2. Referral Code
3. Plan Code
4. Diagnosis Code
5. Specialist Physician Provider ID
6. Referring Physician Provider ID
7. Cost of Reimbursement
8. Patient Zip Code
9. PCP Zip Code
10. Referring Physician Zip Code
11. Patient Age
12. Patient Gender
13. ICN (define)
14. Date of visit—Date of claim?
15. Billing Physician NPI
16. Referring Physician NPI
17. Referring Provider Name
18. CPT procedure Code
19. Procedure Cost
20. Number of Procedures
21. Total Charge
22. Amount Paid
23. Place of Service
24. ICD10 Diagnosis code
25 ICD10 Diagnosis code Preauthorization
26. Billing Provider NPI

Once the Authorized User has uploaded the claims data, the system will analyze the uploaded data as follows:
1. Checks for a valid .CSV file according to the required fields. If the file is invalid, the user gets a message “Attachment content type is invalid. Attachment is invalid.”
2. Checks for duplicate data entries. If the system recognizes an existing row in .CSV file, then this row should be ignored. Only new rows will be successfully imported from .CSV file.
3. After updating “Claims” database table, the system will run update algorithm for the “Referral”table according to the following Rules:
   Member ID on claim=Member ID on pending referral
   AND
   Physician NPI on claim=NPI of a physician referred to OR if referral was to a physician group then the NPIs of the physicians in the referred group
   AND
   CPT code on claim=one of the CPT codes in the procedure family object of the referral
   AND
   Date of claim between a referral date and referral date +90 days

If there is a match, the “Referral”table will be updated by the following fields:
1. the status should change to “Completed”
2. the completed claim should become a pointer to the matching claim
3. the claim date should become the date of the matching claim.

Accordingly, following the above analysis, the system updates the referral with the new data and provides the Authorized User with the following information:
Number of rows uploaded
Number of matches found
Number of Rows Rejected Due to Duplication.

If there is a match the status is changed to Completed. The Completed Claim will become a pointer to the matching claim and the claim date will become that date of the matching claim. In addition, if on that date when new claims are uploaded there exist pending referrals that are 90 or more days old and not a match, their status will be changed to “Expired”. There is a high likelihood these referrals will never be matched to a claim. However, there is an off chance the user may update a matching claim at some later point in time, so even expired claims will be compared to newly uploaded claims.

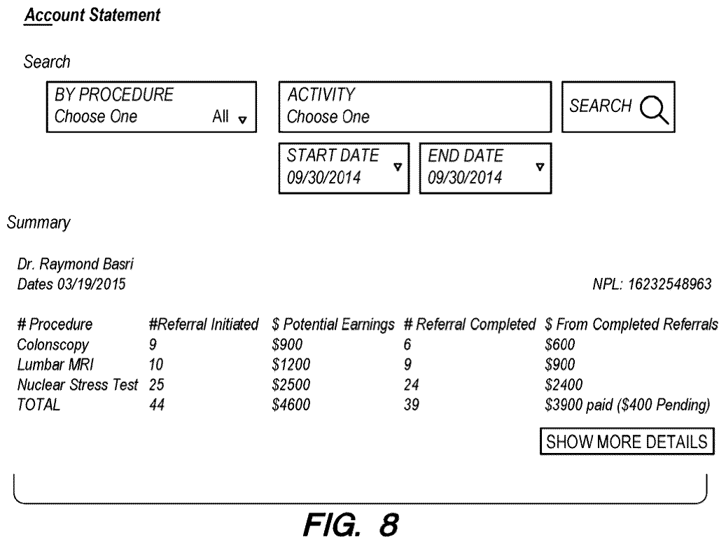

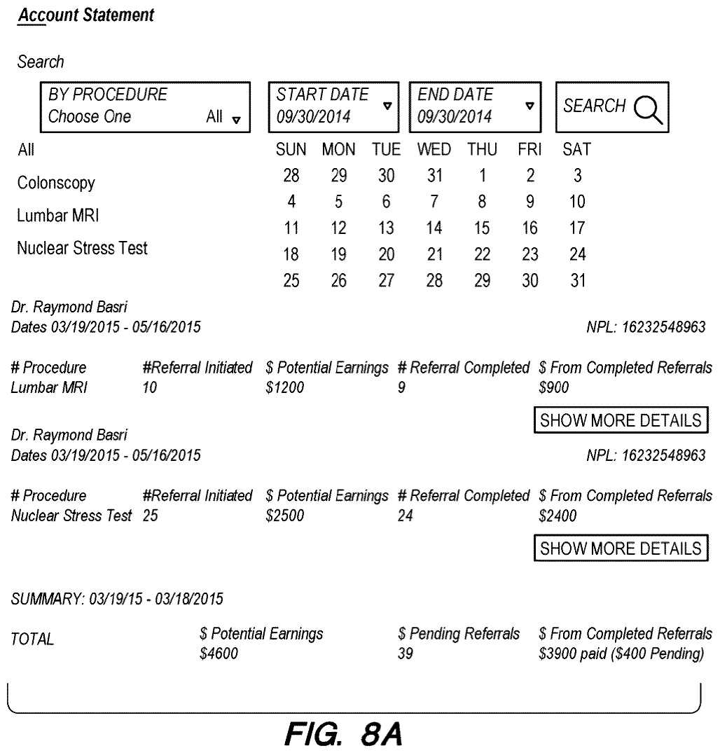

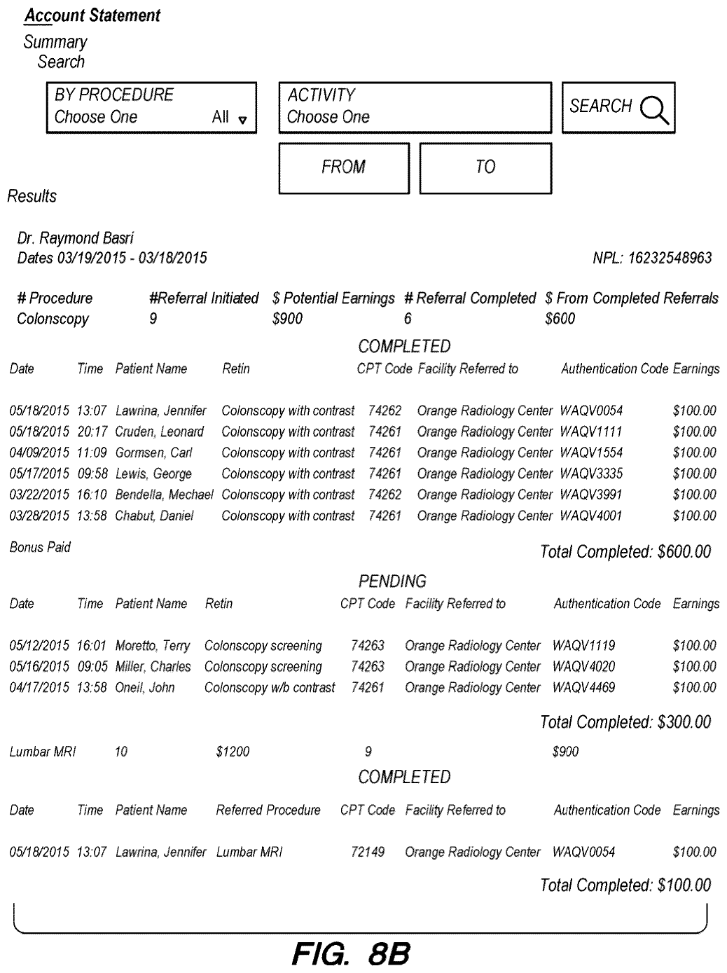

An Authorized User may also view all referral objects and see the current status of all referrals made by an individual Physician using filters by procedure family and date. As depicted in FIG. 8. As can be seen in FIG. 8, the Authorized User searched for all procedures between a particular date range. For such a query, the System makes the following steps:
1. Search the database by all procedures and specified date range.
2. Show all the found results to user as a default table, FIG. 8, with the total sum by all values in the last row.

FIG. 8A depicts the Procedure Families available to the Authorized User for the query. Each referring physician's account statement will contain information unique to that physician's referrals. FIG. 8B depicts a query for all dates and all procedure families.

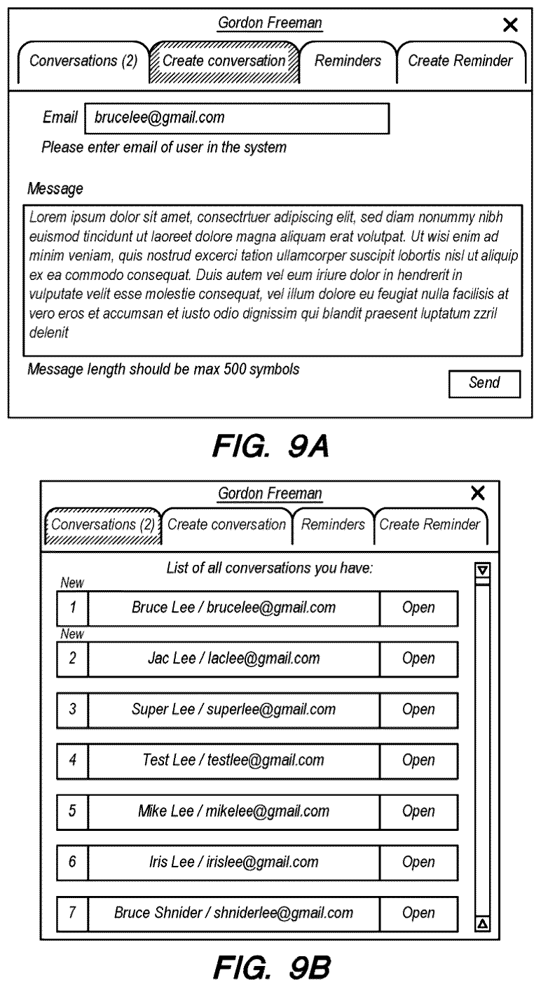

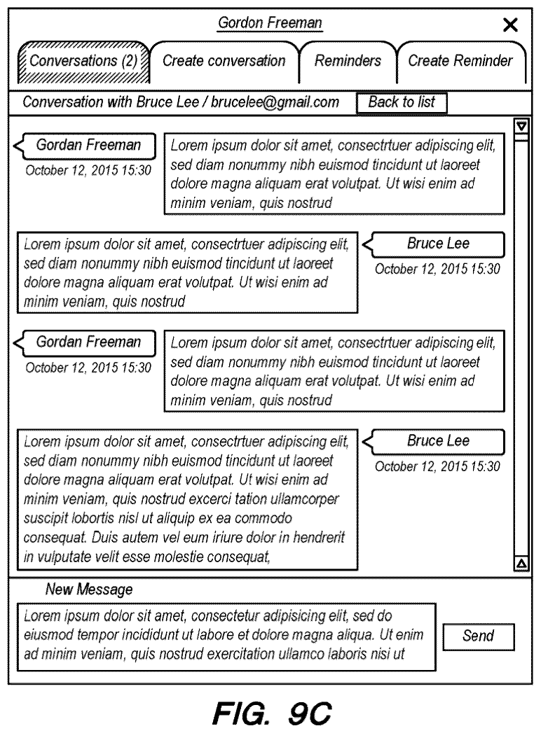

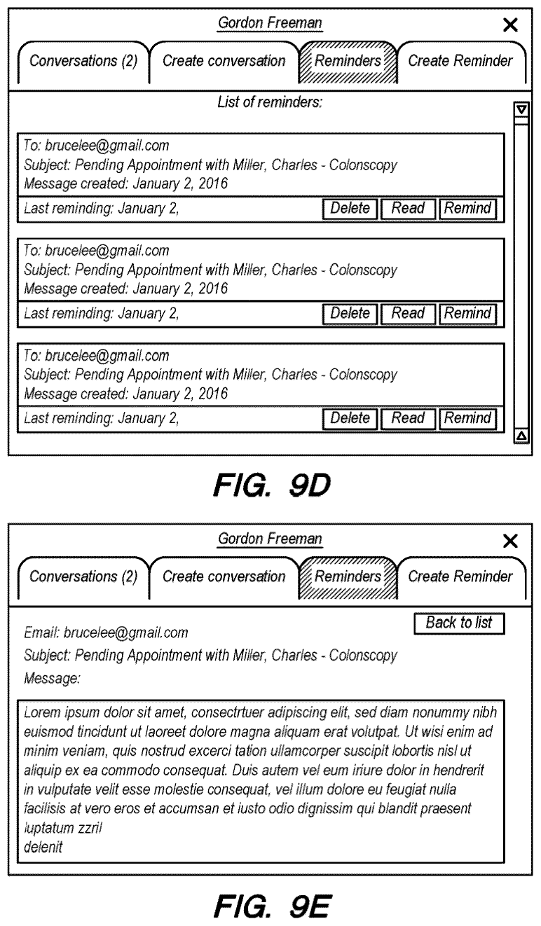

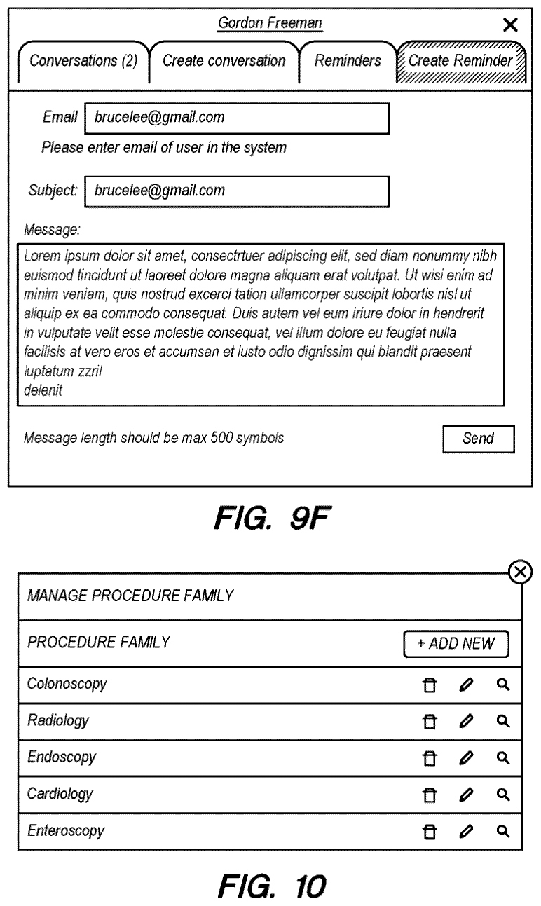

In the event an Authorized User desires to communicate with or send a reminder to a physician or vice versa, the parties may utilize the Messenger function, as depicted in FIGS. 9A, 9B, 9C, 9D, 9E, 9F.

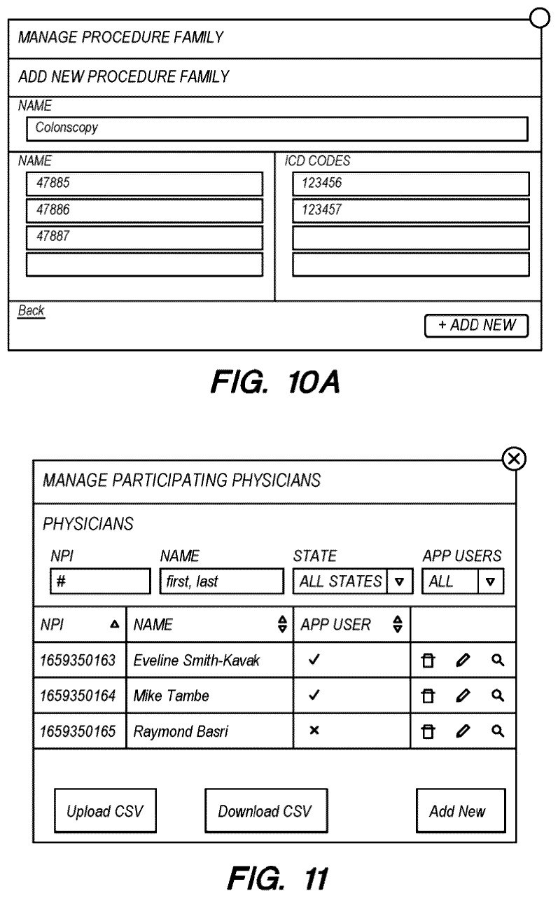

An Authorized User is also able to modify Procedure Families using the Account Management dashboard. By clicking on Procedure Family, the Authorized User is redirected to the screen as depicted in FIG. 10. The Authorized User may click on the “trash can” to delete, the “pencil” to edit, or the “magnifier” to see a preview of the Procedure Family Selected. In addition, an Authorized User may add a new Procedure Family by clicking on “+ADD NEW”. When adding a new Procedure Family, the Authorized User must enter the CPT codes associated with that Family, as depicted in FIG. 10A.

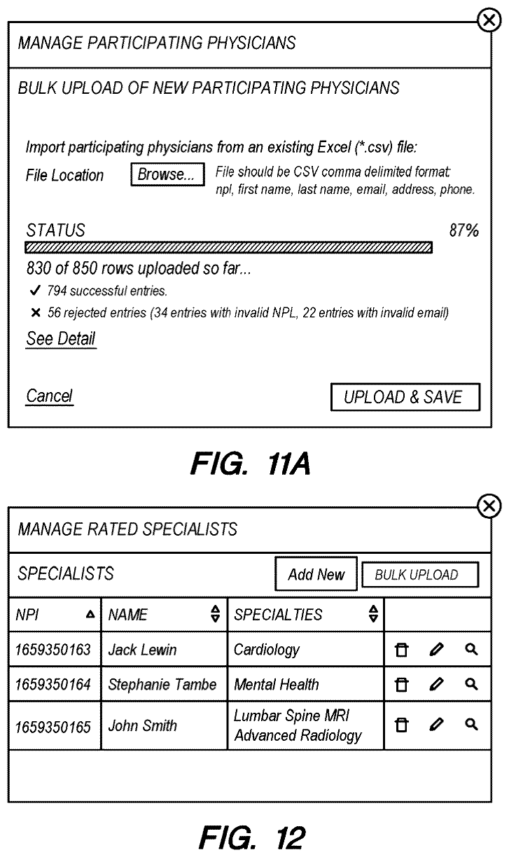

The Authorized User may also manage the Participating Physicians database using the Account Management dashboard, as depicted in FIG. 11. A Physician may be located by NPI, Name or State. Further, physicians records may be deleted (trash can icon), edited (pencil icon) or viewed (magnifying glass icon), as desired. The Authorized User may also upload new physician reads using the “Upload CSV” icon. This upload may be a mass upload, FIG. 11A, or individual. Further the Authorized user may download the record of a particular physician.

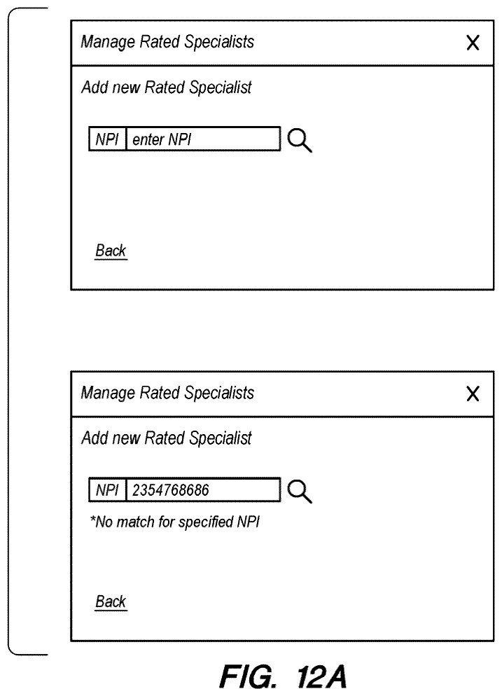

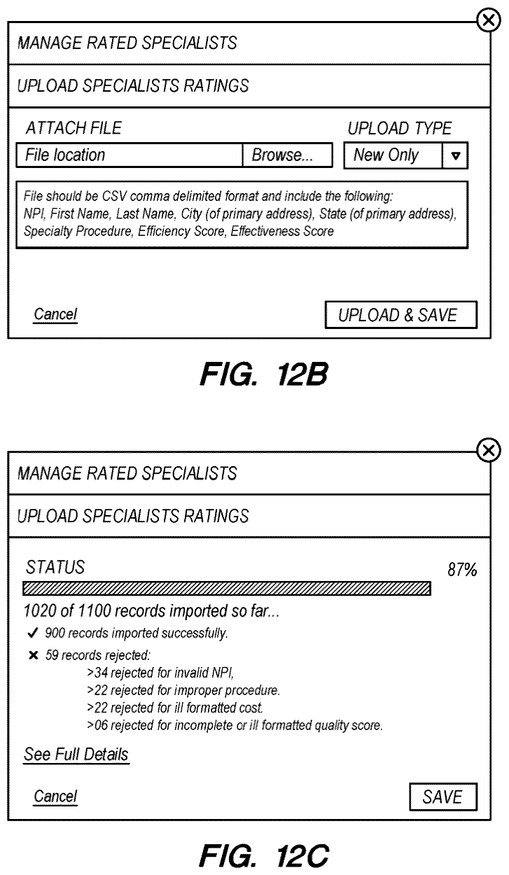

An Authorized User may manage, view and edit Specialists by their market rate, referred to as Rated Specialists in the Account Management dashboard, as depicted in FIG. 12. The procedure for managing the Rated Specialists is similar to the procedure for managing Participating Physicians. In order to add a new rated specialist individually, the Authorized User enters the NPI associated with that individual, FIG. 12A. Further, the Authorized user may upload specialist by claims data using “BULK UPLOAD” and attaching the relevant file, FIGS. 12B and 12C.

Once the claims data set is specified and properly uploaded (if using new claims), the system will do the following post processing of the data:
1. For each claim, map the CPT code into a procedure family. (the mapping table is the procedure family object)
2. Group the data by a physician and procedure family to create a list of all unique physician-procedure family pairs.
3. Ensure all physicians in the claim data have an NPI that matches the global provider database
4. Derive the median cost of all claims for this provider, p, and procedure family, f, (cp, f)
5. Derive the median cost of all claims for this procedure family, f, within 20 miles of provider p. (cp, f)
6. Define market rate as (cp, f)
7. Define efficiency score of this provider as (cp, f) / (cp, f).

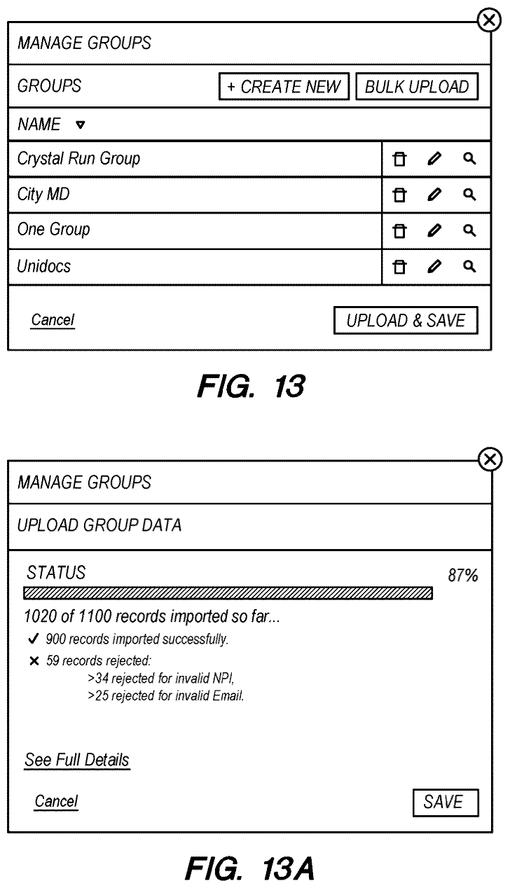

An Authorized User may also create physician groups, the linkages between groups and participating physicians, as well as the links between groups and rated specialists using “Physician Groups” in the Account Management dashboard. This allows the Authorized User to analyze data on a group level, e.g., what is an average cost for this group. The system allows the Authorized User to see a list of all existing physician groups in the system, FIG. 13, and also allows the Authorized User to add a new physician group, individually or as a bulk upload. A new Physician Group to be uploaded must be a. CSV file containing the following data:
1. Physician NPI (required)—the NPI of a physician in the rated specialist or participating physician group.
2. Tax identification number (required)—it will be a true unique identifier of the group.
3. Name (optional)—the unique name of the physician group
4. Street Address 1 (optional)—street address 1
5. Street Address 2 (optional)—street address 2
6. City (optional)—the city
7. State (optional)—the US state
8. ZIP code (optional) the US postal code of the group
9. Contact Name (optional)—string, does not have to be a physician, so no data validation
10. Email address (optional)—a central email for this group
11. Phone Number (optional)—a central phone number for the group

Once the user uploads the specified. CSV, the following data validation is required:
1. Validate that all uploaded NPIs match an existing NPI in the users participating provider (global data set) and rated specialist data.
2. Validate all tax ID numbers are 9 digit integers. (hyphens, periods, and commas are allowed in input and should be ignored when processing. E.g., 111-11-1111 is acceptable and should be imported as 111111111). The user always ascribes the same group info (columns 3-11: name, address field, emails, and phone numbers) to each tax id.

After this data validation step, the system should display a dialogue box that outputs the results of the upload such as: “850 rows uploaded. 34 rejected, because NPI does not match an existing participating physician or rated specialist. 22 rejected because tax id invalid. 14 rejected, because inconsistent attributes of the group. 780 successfully uploaded”, FIG. 13A.

1. A computer-based method of using data markers in a health insurance entity claims database of a health insurance entity to assist in tracking episodes of care to assist in generating referrals of medical specialists to a referring user, the method comprising:
(1) in a backend process:
accessing a health insurance entity claims database of a health insurance entity storing historical claims of patients;
evaluating the historical claims to retrospectively estimate historical episodes of care associated with each core medical procedure, each retrospectively estimated historical episode of care including a historical core medical procedure and historical subsequent medical events retrospectively estimated to be associated with the historical core medical procedure; and
uniquely marking each retrospectively estimated historical episode of care in the health insurance entity claims database with a respective data marker, the unique marking including adding the respective data marker into a respective field that is capable of being searched and filtered based on the respective data marker stored therein;
(2) in a first runtime process:
providing, by a computing system, a referral system interface for enabling a referring user to request a particular medical procedure for a particular patient and for making a referral of a particular medical specialist to assist with the particular medical procedure;
identifying a set of medical specialists from a medical specialist database who are able to perform the particular medical procedure;
accessing the health insurance entity claims database to locate retrospectively estimated historical episodes of care and if available one or more actual historical episodes of care by one or more of the set of medical specialists associated with a same medical procedure as the particular medical procedure;
using predictive analytics on a subset of the retrospectively estimated historical episodes of care and when available on the one or more actual historical episodes of care to generate an expected episode of care for each medical specialist of the set of medical specialists for the particular medical procedure, the expected episode of care for each medical specialist of the set of medical specialists including the particular medical procedure and expected subsequent medical events expected based on the expected episode of care for the medical specialist;
generating an expected cost for the expected episode of care for each medical specialist of the set of medical specialists;
generating a list of candidate medical specialists from the set of medical specialists based on the expected cost of the expected episode of care for each medical specialist of the set of medical specialists;
transmitting the list of candidate medical specialists to the referring user;
receiving selection of the particular medical specialist; and
generating a particular data marker to track an actual episode of care of the particular medical specialist in the health insurance entity claims database of the health insurance entity;
using the particular data marker to mark iteratively an actual claim for the particular medical procedure and each actual claim for the one or more actual subsequent medical events to dynamically track the actual episode of care of the particular medical specialist for the particular medical procedure in the health insurance entity claims database; and
(3) in a second runtime process:
without requiring the backend process (1), repeating the first runtime process (2) however for a second referring user to request a second particular medical procedure for a second particular patient and for making a second referral of a second particular medical specialist to assist with the second particular medical procedure.

2. The method of claim 1, wherein each data marker is a unique string of numbers.

3. The method of claim 1, further comprising the referring user entering a preferred travel distance for the particular medical procedure to filter the list of candidate medical specialists.

4. The method of claim 1, further comprising entering a cost variance for the particular medical procedure by the particular medical specialist.

5. The method of claim 1, further comprising:
querying a patient medical records database of patient medical records of patients to locate the particular patient's medical record; and
using the particular patient's medical record to access a health insurance coverage records database of health insurance coverage records of the patients to access the particular patient's health insurance coverage record.

6. The method of claim 5,
wherein each patient medical record includes at least a first name, a last name, a date of birth and a phone number;
wherein each health insurance coverage record includes at least a type of policy, network of medical service providers associated with the policy, procedures covered by the policy, coverage limits of the policy, deductibles associated with the policy, and amounts paid to date for the patient for the policy; and
wherein the medical specialist database includes a name of each medical specialist, at least one specialty service of the medical specialist, a location of the medical specialist, and hours of operation of the medical specialist.

7. The method of claim 1, wherein each historical claim includes at least a procedural terminology code for a procedure, amount paid by the health insurance entity for the procedure and a location of the medical specialist for the medical procedure.

8. The method of claim 1, further comprising calculating a payment to the referring user based on the actual cost of the actual episode of care.

9. The method of claim 8, wherein the payment is generated relative to a calculated market amount.

10. The method of claim 9, wherein the calculated market amount is based on cost variance for different medical specialists of the same medical procedure within a given distance from the particular patient.

11. The method of claim 1, wherein the computing system is deployed by a health insurance entity with access to the health insurance entity claims database.
